(19) United States
(12) Patent Application Publication
(10) Pub. No.: US 2026/0034320 A1
(43) Pub. Date: Feb. 5, 2026

(54) SYSTEM, DEVICE, AND METHODS FOR ASSESSING THE FIT AND PERFORMANCE OF PERSONAL, DISPOSABLE RESPIRATORS

(71) Applicant: University of Maryland, College Park, College Park, MD (US)

(72) Inventors: Lester W. SCHULTHEIS, Sykesville, MD (US); Quinton BURKE, Fallston, MD (US); John Robertson RZASA, University Park, MD (US)

(21) Appl. No.: 19/283,657

(22) Filed: Jul. 29, 2025

Related U.S. Application Data
(60) Provisional application No. 63/678,741, filed on Aug. 2, 2024.

(51) Int. Cl. A61M 16/00

(52) U.S. Cl. CPC A61M 16/0003; A61M 2205/15

(57) ABSTRACT

In accordance with the present disclosure, a method for detecting leakage from a disposable respirator, includes: placing a respirator comprising an infrared light source on a user's face; capturing a baseline image of the user's face with the IR light source in an inactive state using an imaging device configured to detect infrared radiation; activating the IR light source to emit infrared light toward regions proximate to a sealing interface between the respirator and the face; capturing a bandpass-filtered image of the user's face with the IR light source in an active state using the imaging device; processing the bandpass-filtered image to generate a processed filtered image, the process-filtered image isolating reflected IR light indicative of leakage at the sealing interface; generating a threshold image highlighting regions where IR light has escaped through gaps in the sealing interface; and analyzing the threshold filtered image to determine a measure of leakage.

### CROSS-REFERENCE TO RELATED APPLICATION AND CLAIM OF PRIORITY

This application claims the benefit of, and priority to, U.S. Provisional Patent Application No. 63/678,741, filed on Aug. 2, 2024, the entire contents of which are hereby incorporated herein by reference.

### TECHNICAL FIELD

The present disclosure relates generally to the field of filtration and respiratory protection systems. More specifically, the present disclosure provides systems, devices, and methods for assessing and improving the fit and performance of personal, disposable respirators using infrared imaging, chemical indicators, and anatomical sealing enhancements.

### BACKGROUND

Respiratory protective devices such as N95 respirators are widely used to reduce exposure to airborne contaminants, including infectious agents, dust, and other hazardous particulates. These devices function by forming a seal around the user's nose and mouth and filtering inhaled air through specialized materials. When properly fitted, respirators provide effective protection in occupational, clinical, and public health settings. However, the efficacy of such devices is highly dependent on the quality of the face-to-mask seal.

One persistent challenge with disposable respirators is the inability of many users to achieve a proper fit. Even slight gaps between the respirator and the skin can allow unfiltered air to bypass the filtration media entirely. Fit testing protocols developed by OSHA, such as quantitative testing with aerosolized particles or qualitative methods using saccharin (or other taste sensitive chemicals), require specialized equipment, trained personnel, and controlled environments. These constraints make standardized fit testing impractical or inaccessible in many non-occupational or low-resource settings.

A second issue arises during extended or repeated use of a respirator. Users frequently don and doff masks during shifts or wear them for prolonged periods. Over time, the fit may degrade due to wear, facial movement, or material fatigue, often without the user's awareness. Conventional respirators offer no timely feedback mechanism to alert users of newly developed leaks, resulting in unintentional exposure to harmful airborne agents.

A third challenge involves anatomical variability. Commercially available respirators are typically mass-produced in a limited range of shapes and sizes and may not conform adequately to users with facial structures that fall outside the population averages used in respirator design databases. In particular, individuals with prominent nasal bridges or atypical facial contours may experience persistent leakage in areas like the nasal root, where sealing is difficult to achieve with standard components such as malleable nose wires.

Therefore, there remains a need for systems, devices, and methods that enable rapid, accessible, and real-time assessment of respirator fit without requiring specialized infrastructure, including facilities, personnel, and training, that detect emergent leaks during use, and that accommodate facial anatomical variability to improve seal integrity.

### SUMMARY

In accordance with the present disclosure, a method for detecting leakage from a disposable respirator, includes: placing a respirator comprising an infrared (IR) light source on a user's face; capturing a baseline image of the user's face with the IR light source in an inactive state using an imaging device configured to detect infrared radiation; activating the IR light source to emit infrared light toward regions proximate to a sealing interface between the respirator and the face; capturing a bandpass-filtered image of the user's face with the IR light source in an active state using the imaging device; processing the bandpass-filtered image to generate a processed filtered image, the process-filtered image isolating reflected IR light indicative of leakage at the sealing interface; generating a threshold image highlighting regions where IR light has escaped through gaps in the sealing interface; and analyzing the threshold filtered image to determine a measure of leakage based on spatial distribution or intensity of the escaped light.

In an aspect of the present disclosure, the infrared light source may comprise one or more infrared light-emitting diodes (IR LEDs) configured to emit light in a wavelength range of about 850 to 940 nanometers.

In an aspect of the present disclosure, the wavelength range of approximately 850 to 940 nanometers may be selected to minimize absorption by melanin, thereby enabling consistent detection of infrared reflectance across users with varying skin pigmentation.

In an aspect of the present disclosure, the imaging device may include a bandpass optical filter configured to selectively transmit infrared light in the same wavelength range as the IR light source.

In an aspect of the present disclosure, the method may further include capturing an unfiltered image of the user's face with the IR light source in an active state using the imaging device.

In an aspect of the present disclosure, the method may further include calculating a leakage percentage by dividing a number of pixels above a defined intensity threshold within the highlighted regions by a total number of pixels within the highlighted regions.

In an aspect of the present disclosure, the method may further include displaying the processed filtered image on a user interface during use to visually indicate leakage location and severity.

In an aspect of the present disclosure, the IR light source is configured within the inside surface of the respirator.

In an aspect of the present disclosure, the measure of leakage may include a computed fit score, leak index, or qualitative diagnostic label based on predefined criteria.

In an aspect of the present disclosure, the method may further include detecting infrared light leakage from a plurality of respirators worn by multiple users within a shared field of view; and concurrently evaluating a measure of leakage for each of the plurality of respirators.

In accordance with the present disclosure, a system for detecting leakage from a disposable respirator, includes: an infrared (IR) light source located within a respirator and configured to emit IR light toward a sealing interface between the respirator and a user's face; an imaging device configured to capture a baseline image of the user's face with the IR light source in an inactive state and a bandpass-filtered image with the IR light source in an active state; a processor; and a memory, including instructions stored thereon, which when executed by the processor cause the system to process the bandpass-filtered image to identify a zone of illumination, wherein the zone of illumination is isolated IR light indicative of leakage through the sealing interface; generate a processed filtered image highlighting regions of IR light escape; and analyze the processed filtered image to determine a measure of leakage based on the spatial distribution or intensity of the escaped IR light.

In an aspect of the present disclosure, the infrared (IR) light source may comprise one or more infrared light-emitting diodes (IR LEDs) configured to emit light in a wavelength range of about 850 to 940 nanometers.

In an aspect of the present disclosure, the wavelength range of approximately 850 to 940 nanometers may be selected to minimize absorption by melanin, thereby enabling consistent detection of infrared reflectance across users with varying skin pigmentation.

In an aspect of the present disclosure, the imaging device may include a bandpass optical filter configured to selectively transmit infrared light in the same wavelength range as the IR light source.

In an aspect of the present disclosure, the processor may be further configured to perform pixel-wise processing between the baseline image and an unfiltered image to generate a differential image representing localized IR reflectance changes, wherein the unfiltered image is of the user's face with the IR light source in an active state.

In an aspect of the present disclosure, the processor may be further configured to apply an intensity threshold to the processed filtered image to generate a threshold image highlighting regions exceeding a predefined leakage threshold.

In an aspect of the present disclosure, the processor may be further configured to calculate a leakage percentage by dividing a number of pixels above a defined intensity threshold within the zone of illumination by a total number of pixels within the zone of illumination.

In an aspect of the present disclosure, the IR light source may be located along an inner surface of the respirator adjacent to the sealing interface.

In an aspect of the present disclosure, the measure of leakage may comprise a computed fit score, leak index, or qualitative diagnostic label based on predefined criteria.

In an aspect of the present disclosure, the processor may be further configured to analyze the processed image independently of user skin pigmentation by leveraging spectral insensitivity of the selected IR wavelength band to melanin content.

### BRIEF DESCRIPTION OF THE DRAWINGS

A better understanding of the features and advantages of the present disclosure will be obtained by reference to the following detailed description that sets forth illustrative embodiments, in which the principles of the present disclosure are utilized, and the accompanying drawings of which:

FIG. 1 illustrates a diagram of a system for detecting leakage from a disposable respirator, including an IR LED respirator system, an imaging system, and a controller, in accordance with the present disclosure;

FIG. 2 illustrates a block diagram of the controller of the system of FIG. 1, in accordance with the present disclosure;

FIG. 3A illustrates a baseline image of a user wearing the IR LED respirator system of FIG. 1, in accordance with the present disclosure;

FIG. 3B illustrates an unfiltered image of the user wearing the IR LED respirator system of FIG. 1, in accordance with the present disclosure;

FIG. 3C illustrates a bandpass-filtered image of the user wearing the IR LED respirator system of FIG. 1, in accordance with the present disclosure;

FIG. 4A illustrates a subtracted image of the user wearing the IR LED respirator system of FIG. 1, in accordance with the present disclosure;

FIG. 4B illustrates a threshold image of the user wearing the IR LED respirator system of FIG. 1, in accordance with the present disclosure;

FIG. 5 is a graphical illustration of the correlation between fit factor and percentage area of light identified by the system of FIG. 1, in accordance with the present disclosure;

FIGS. 6A and 6B illustrates an exemplary embodiment of detecting leakage across a range of simulated skin pigmentation levels by the system of FIG. 1, in accordance with the present disclosure;

FIG. 7 is a flowchart of an exemplary method for detecting leakage from a disposable respirator, in accordance with the present disclosure;

FIG. 8 illustrates a colorimetric leak detection system, in accordance with the present disclosure;

FIGS. 9A and 9B illustrate an exemplary embodiment of an indicator of the system of FIG. 8, in accordance with the present disclosure; and

FIG. 10 illustrates a sealing enhancement system integrated into a respirator, in accordance with the present disclosure.

### DETAILED DESCRIPTION

The present disclosure relates generally to the field of respiratory protection and filtration performance. More specifically, the present disclosure relates to systems and methods for real-time, self-contained, and non-invasive assessment of respirator fit and leakage using infrared imaging, chemical indicators, and anatomically adaptive sealing elements to enhance the protective function of disposable respirators across diverse use environments.

Although the present disclosure will be described in terms of specific examples, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the novel features illustrated herein, and any additional applications of the principles of the present disclosure as illustrated herein, which would occur to one skilled in the relevant art and having possession of this disclosure, are to be considered within the scope of the present disclosure.

Conventional methods for respirator fit testing, such as OSHA's quantitative fit factor testing using aerosolized particles, require specialized equipment, controlled environments, and trained personnel. These limitations render such methods impractical for receiving timely feedback in field use, continuous monitoring, or rapid assessment in dynamic operational settings. Moreover, qualitative fit testing methods are inherently subjective and unable to quantify seal degradation over time. The present disclosure addresses these technical challenges by providing a compact system for detecting leakage in disposable filtering facepiece respirators using near-infrared (IR) reflectance imaging and automated image analysis. The system integrates an IR light source internal to a respirator, an external imaging device, and a controller configured with onboard processing capabilities for real-time detection, quantification, and visualization of air leakage across the sealing interface. By using IR wavelengths that are minimally affected by melanin, the system ensures consistent performance across users of varying skin pigmentation, thereby mitigating bias and reducing the need for individual calibration. This allows for rapid, non-invasive, and scalable respirator fit assessment suitable for deployment in occupational, clinical, and emergency environments without the infrastructure demands of traditional fit testing modalities. In some embodiments, the system further includes a sealing enhancement system comprising biocompatible adhesive strips that conform to the user's face and improve fit across a wider range of facial geometries. In another embodiment, a passive colorimetric leak detection system allows for persistent leak monitoring through visible pH-based color change in response to exhaled breath, facilitating intuitive and continuous fit verification during respirator use.

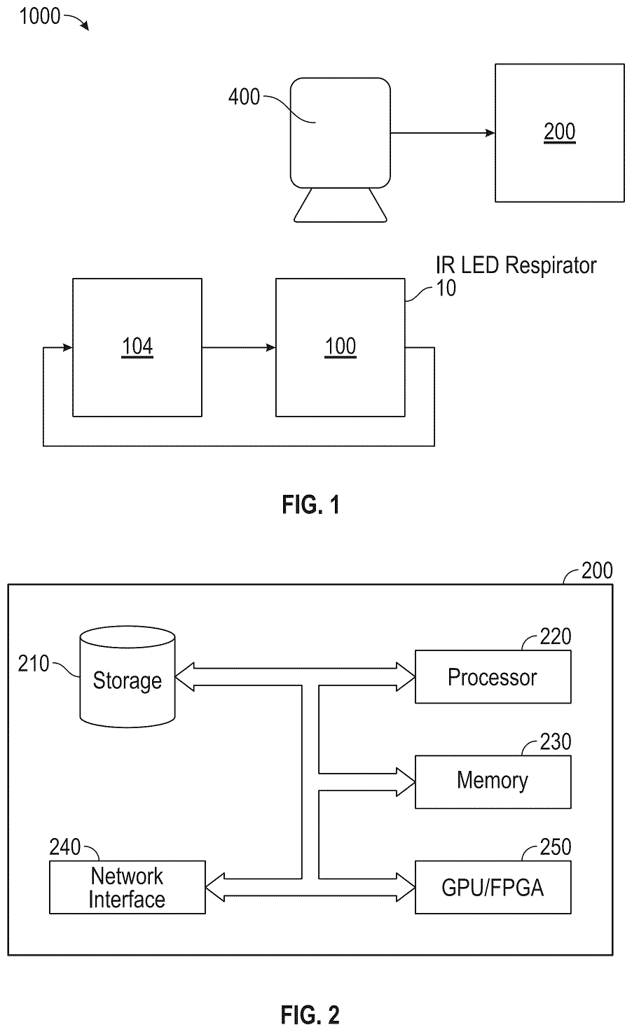

Referring to FIGS. 1-5, an aspect of the present disclosure is directed to systems and methods for assessing the fit of a filtering facepiece respirator using infrared (IR) light reflectance to detect leakage at the seal interface. The system allows for rapid, non-subjective, and field-deployable identification of mask leakage without requiring specialized environments or equipment.

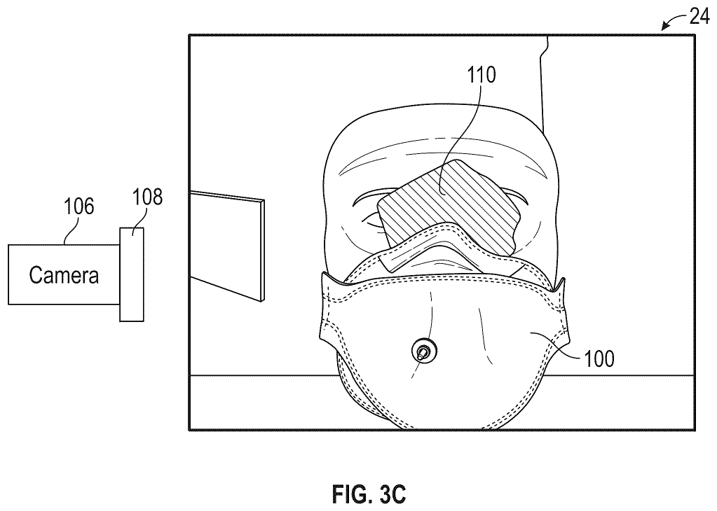

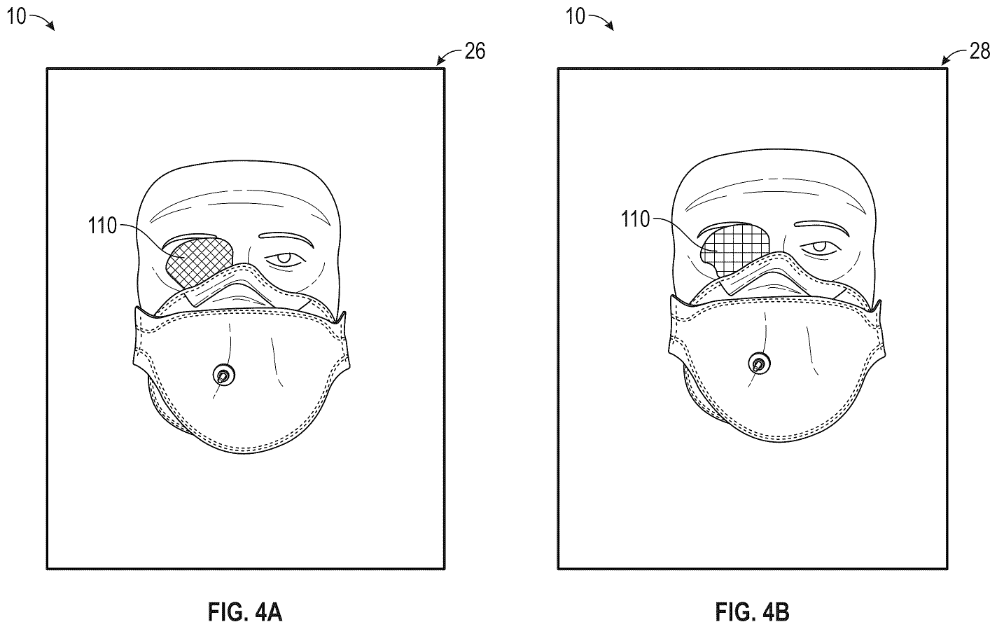

FIG. 1 illustrates a system 1000 for detecting leakage from a disposable respirator. The system 1000 includes an infrared (IR) light emitting diode (LED) respirator system 10, imaging system 400 configured to capture one or more images of the user using the IR LED respirator system 10, and a controller 200. The imaging system 400 may include a camera 106 (FIG. 3A) fitted with a bandpass optical filter to isolate light within the emitted IR spectrum. The captured image data is transmitted to a controller 200, which includes at least one processor and memory, and is configured to analyze the images to detect light escaping through gaps in the seal of the respirator 10. The controller 200 performs image processing, for example subtraction, between a baseline image (IR off) and an illuminated image (IR on), applies threshold filters to identify leakage regions, and calculates the percentage area of light leakage based on pixel intensity above a predetermined threshold. The system 1000 may further include a user interface or output module for displaying leakage information to the user or a supervisor. The system 1000 is described in more detail below with reference to FIGS. 3 and 4.

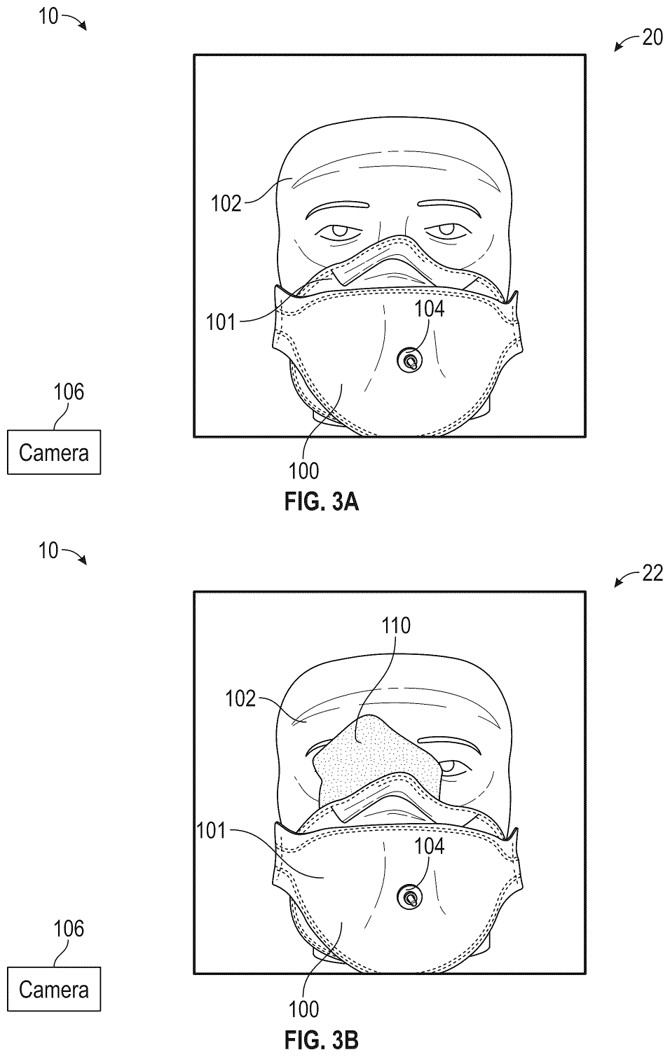

Referring now to FIG. 2, a controller 200 is illustrated which may be part of or in communication with the respirator leakage detection system. In one embodiment, the controller 200 includes one or more processors 220, memory 230, and storage 210. The processor 220 is configured to execute instructions for image processing operations including baseline processing, infrared threshold filtering, pixel summation, and determination of leakage area percentage. The memory 230 temporarily stores image data captured by the camera during both baseline (e.g., FIG. 3A) and illuminated (e.g., FIG. 3B) states. The processor 220 may compare the respective pixel intensities to isolate light leakage above a predefined threshold.

The controller 200 may also include a database 210 to retain baseline image profiles, patient/user identifiers, time-stamped leakage events, and processing parameters. In some aspects, the controller may further include a network interface 240 for transmitting leakage data to remote systems (e.g., occupational health monitoring platforms) or logging usage patterns over time. In certain configurations, a graphical processing unit (GPU) or field-programmable gate array (FPGA) 250 may be incorporated to accelerate image processing and threshold analysis across multiple users or in real-time applications. These components may be embedded in a portable edge device or hosted on a remote cloud server depending on system constraints.

Referring now to FIG. 2, exemplary components in the controller 200 in accordance with aspects of the present disclosure include, for example, a database 210, one or more processors 220, at least one memory 230, and a network interface 240. In aspects, the controller 200 may include a graphical processing unit (GPU) 250, which may be used for processing machine learning models.

Database 210 can be located in storage. The term “storage” may refer to any device or material from which information may be capable of being accessed, reproduced, and/or held in a chemical-based digital form, or an electromagnetic or optical form for access by a computer processor. Storage may be, for example, volatile memory such as RAM, non-volatile memory, which permanently holds digital data until purposely erased, such as flash memory, magnetic devices such as hard disk drives, and optical media such as a CD, DVD, Blu-ray disc, or the like. In various embodiments, data may be stored on the controller 200, including, for example, user preferences, historical data, and/or other data. The data can be stored in database 210 and sent via the system bus to the processor 220.

As will be described in more detail later herein, the processor 220 executes various processes based on instructions that can be stored in the server memory 230 and utilizing the data from the database 210. The illustration of FIG. 2 is exemplary, and it will be understood by persons skilled in the art that other components may exist in a controller 200. Such other components are not illustrated in FIG. 2 for clarity of illustration.

FIGS. 3A-3C illustrate an aspect of IR LED respirator system 10 for detecting leakage in a disposable respirator using infrared (IR) light reflectance. The IR LED respirator system 10 includes a filtering facepiece respirator 100, an infrared (IR) light emitting diode (LED) 104 (“IR LED 104”), and a camera 106. The respirator 100 is worn over the face 102 of a user, such as a human subject or an anthropometric model. The respirator 100 includes a sealing surface 101 that defines the contact interface between the respirator and the user's face 102. This sealing surface 101 is configured to contour around the nasal bridge, cheekbone, and chin regions tightly to seal the airways and minimize air leakage. As illustrated, the IR LED 104 is configured within the inner cavity or inner boarder margin of the respirator 100. In some aspects, the IR LED 104 is oriented to emit light toward the user's face 102 and the sealing surface 101. In other aspects, the IR LED 104 may be configured to emit light at a wide-angle or omnidirectionally to ensure coverage of potential air leakage zones.

The IR LED respirator system 10 utilizes infrared light reflectance rather than relying on thermal imaging or temperature gradient measurement. By actively emitting infrared light and capturing its reflection from leakage sites, the system delivers consistent and reliable detection of seal discontinuities. This reflectance-based method is inherently resilient to environmental temperature fluctuations, user activity, or skin temperature variation, offering greater specificity and accuracy in identifying air leakage compared to passive thermal systems.

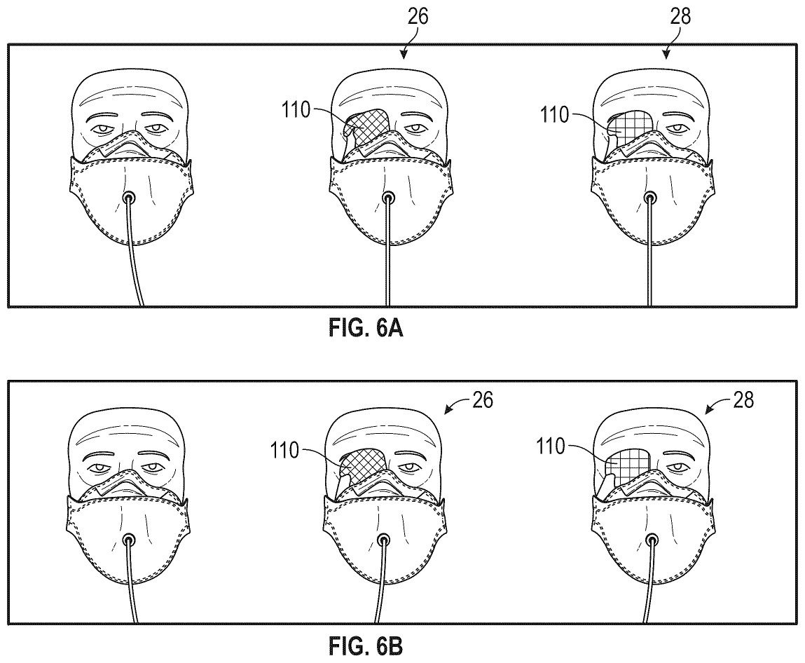

The IR LED 104 may be positioned proximate to anatomical regions that are most prone to leakage, including those underlying the sealing surface 101, such as along the nasal bridge, the nasolabial folds, and the mid-cheekbone region. In other aspects, the IR LED 104 may be positioned to illuminate the entire inside of the respirator 100. In some embodiments, the IR LED 104 is affixed directly to the interior mask surface, while in other embodiments it may be suspended, embedded, or integrated into the mask structure. The IR LED 104 is configured to emit narrowband IR light at a wavelength of approximately 850 nm to 940 nm, which is invisible to the human eye but detectable by an infrared-sensitive imaging camera 106. This spectral range is selected due to its high transmissibility through air gaps and its insensitivity to skin pigmentations, making it well suited for consistent detection regardless of user ethnicity or complexion. The infrared wavelength range utilized by IR LED respirator system is insensitive to melanin content, allowing it to detect leakage consistently across users with different skin tones. As shown in FIG. 6B, reflectance measurements obtained using this infrared configuration remain consistent in manikin models representing different pigmentation levels, without the need for calibration or signal compensation.

As shown in FIG. 3A, the respirator 100 is placed on the face 102 of a user with the IR LED 104 in an inactive or “off” state. A baseline image 20 is captured by the camera 106 in the inactive or “off” state. This image may be used to characterize the ambient lighting conditions, facial surface reflectance, and any internal reflections caused by the mask geometry. At this stage, the camera 106 observes only ambient light and visible reflection from the respirator 100, which may later be digitally compared during image analysis to enhance signal clarity.

Upon activation of IR LED 104, as illustrated in FIG. 3B, infrared light is emitted toward the sealing surface 101. In areas where the sealing surface 101 maintains full contact with the user's face 102, the emitted light is absorbed or internally reflected. However, in regions where the sealing surface 101 does not maintain full contact with the user's face, light escapes through gaps of the sealing surface 101 and reflects off the face 102, producing a localized region of high brightness, or a zone of illumination 110. The zone of illumination 110 is observable on the surface of the user's face 102. The IR-sensitive camera 106, positioned externally and facing the user's face 102, captures an unfiltered image 22 of the zone of illumination 110 during LED 104 active illumination, thereby identifying the locations of respiratory leakage. In some aspects, the zone of illumination 110 in the unfiltered image 22 may be a bright white light surrounded by a purple-colored permitter. The zone of illumination 110 in the unfiltered image 22 may also be indicated by any other synthetic coloration and/or patterning to indicate leakage.

In some aspects, system 1000 may generate a differential image by performing pixel-wise processing (e.g., pixel-wise subtraction) of the baseline image 20 (FIG. 3A), acquired with the IR LED 104 in an inactive state, and the unfiltered image 22 (FIG. 3B), captured with the IR LED 104 in an active state. Both images are acquired under otherwise identical imaging conditions using imaging system 400, including IR-sensitive camera 106. The baseline image 20 serves as a reference representing ambient illumination and natural facial reflectivity in the absence of IR emission, while the unfiltered image 22 contains both ambient light and reflected IR light from leakage regions. By subtracting image 20 from image 22, the system suppresses background signal unrelated to leakage and isolates the additional IR reflectance attributable to sealing discontinuities. The resulting differential image may further enhance visualization of the zone of illumination 110.

As illustrated in FIG. 3C, a bandpass optical filter 108 is coupled to the lens of the camera 106. The bandpass optical filter 108 is configured to selectively transmit light only within the narrow IR spectrum corresponding to the emission band of IR LED 104. This bandpass filter 108 blocks visible light and ambient IR noise, thereby isolating and enhancing the signal produced by escaped IR photons relative to background light or signal noise. The camera 106 with the bandpass optical filter 108 captures a bandpass-filtered image 24. The bandpass-filtered image 24 provides a high-contrast view of the zone of illumination 110, which corresponds to the area of air leakage through discontinuities in the sealing surface 101 of the respirator 100. The zone of illumination 110 in the bandpass optical filter image 24 may be represented by any synthetic coloration or patterning. The use of the bandpass filter 108 improves the signal-to-noise ratio by suppressing incidental ambient lighting. In this embodiment, the camera 106 and bandpass optical filter 108 together generate a spatially resolved intensity map that directly correlates to the distribution of IR light escaping from the respirator 100. Notably, the region of highest intensity, of color or shading, within the zone of illumination 110 may align with the primary leakage pathway, allowing users or clinicians to identify fit deficiencies with precision to a specific anatomical site on the user. The zone of illumination 110 may be irregularly shaped and spatially localized, revealing not only the presence but also the extent and directionality of the leak.

FIGS. 4A and 4B illustrate image processing outputs generated by system 1000 for detecting and visualizing leakage of infrared light from the sealing surface 101 of the respirator 100. FIGS. 4A and 4B illustrate processed visual outputs derived from camera 106 after the acquisition of raw image data under both IR-inactive (baseline image 20) and IR-active conditions (unfiltered image 22 and bandpass-filtered image 24), as previously described in connection with FIGS. 3A and 3B and 3C (respectively).

FIG. 4A illustrates a processed filtered image 26 generated by system 1000, processed filtered image 26 is generated by further processing a bandpass-filtered image 24 captured with the IR LED 104 activated (FIG. 3C). In some embodiments, additional digital masking or segmentation is applied to remove residual IR light that passes through the respirator material itself, yielding processed filtered image 26. The process filtered image 26 is transmitted to controller 200, which includes at least one processor and memory, and optionally a GPU or FPGA, configured to suppress background signal and generate a noise-reduced processed filtered image 26. This differential output enhances contrast by removing non-informative content such as ambient illumination, internal mask reflections, or user skin tone variability. The zone of illumination 110 is revealed within processed filtered 26 as a high-contrast region corresponding to infrared light escaping from leakage sites along the sealing surface 101. In some aspects, the zone of illumination 110 in processed filtered image 26 may be represented by a deep purple color.

Processed filtered image 26 serves as a diagnostic data layer within system 1000, enabling localized identification of regions where infrared light emitted from IR LED 104 has escaped through discontinuities in the sealing surface 101 of respirator 100. The relative intensity distribution of the zone of illumination 110 within processed filtered image 26 corresponds to the magnitude of infrared reflectance detected by camera 106, with higher intensity regions indicating greater leakage exposure. Because the signal in processed filtered image 26 has been selectively isolated using baseline suppression and spectral filtering, the resulting output enhances contrast along sealed and unsealed regions between the respirator 100 and the face 102. In some configurations, system 1000 is configured to transmit or display processed filtered image 26 as a near real-time visual indicator of fit quality. The layer of processed filtered image 26 allows operators or users to interpret the spatial origin, severity, and asymmetry of the leakage event with respect to the sealing geometry of the respirator 100. Additionally, processed filtered image 26 may serve as the basis for further processing within system 1000, including segmentation, quantification, or automated thresholding to produce binary or graded outputs.

In FIG. 4B, system 1000 generates a threshold image 28 from the intensity data present in subtracted image 26. The threshold image 28 is generated by applying a defined pixel intensity threshold value to subtracted image 26, where only pixel values exceeding a predefined intensity threshold are retained. The defined pixel intensity threshold value is selected to correspond to infrared reflectance levels indicative of physiologically and operationally meaningful leakage at the sealing surface 101. Threshold image 28 isolates the most prominent portions of the zone of illumination 110 by filtering out all values below the set detection threshold. The result is a binarized or segmented image, in which high-probability leakage regions are preserved and all background and sub-threshold signals are suppressed. The zone of illumination 110 in the threshold image 28 represents the intensity exceeding a threshold, which may be calibrated to reflect clinically or operationally meaningful leak magnitudes. The zone of illumination 110 in the threshold image 28 may be represented by a vibrant green color. In certain aspects, system 1000 includes logic for quantifying the spatial extent of this zone of illumination by calculating the proportion of pixels above the threshold relative to the total imaged region. This computed percentage represents the percent area of the leak and serves as a key performance metric for assessing respirator fit.

In certain aspects of system 1000, the threshold image 28 is further subjected to quantitative image analysis to compute the relative or absolute area of leakage. Metrics such as pixel count, calibrated physical area, or percentage of the sealing surface 101 may be used to generate a fit quality score, leak index, or visual overlay for interpretability. Additionally, because the thresholding process is performed downstream of signal isolation via image processing (e.g., subtraction) and spectral filtering, the resulting threshold image 28 is robust under variable ambient lighting conditions and remains unaffected by user skin pigmentation, owing to the spectral insensitivity of the selected IR wavelength to melanin absorption.

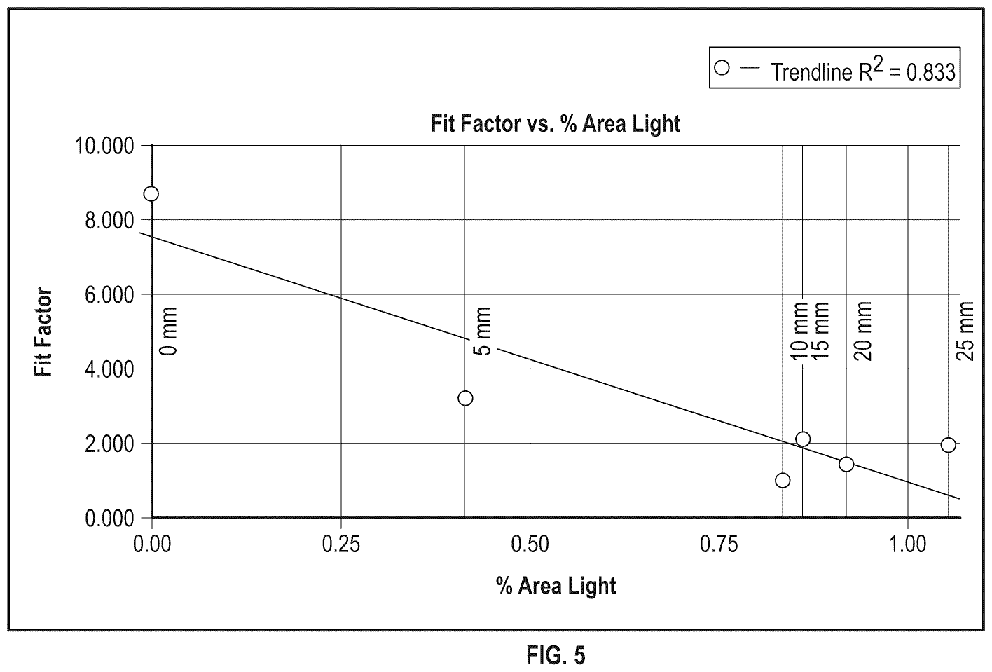

FIG. 5 illustrates a validation output generated by system 1000, demonstrating a correlation between fit factor values and the percent area of light identified in threshold image 28. The data points in FIG. 5 represent a series of controlled experiments in which manual gaps were introduced between the respirator 100 and the face 102 to simulate varying degrees of seal failure. The degree of displacement ranged from 0 mm (tight seal) to 25 mm (maximum gap), with intermediate offsets at fixed increments. For each displacement condition, a fit factor measurement was obtained using a TSI PortaCount instrument under standard OSHA-compliant quantitative fit testing methodology. Concurrently, system 10 acquired IR images using the internal IR LED 104 and processed them through its integrated imaging pipeline to generate threshold image 28, from which the percentage area of light leakage was computed.

The resulting data, as plotted in FIG. 5, illustrates a strong inverse relationship between the computed percent area of light and the corresponding fit factor, with an R² value of 0.833. This trend demonstrates that the percentage area of leakage calculated by system 1000 based on reflectance intensities exceeding a defined threshold in image 28 and serves as a reliable surrogate for conventional aerosol-based fit factor testing. The ability of system 1000 to infer fit factor performance from image-based diagnostics supports its use as a low-cost, non-invasive, and scalable alternative to existing quantitative fit testing infrastructure. Moreover, the system's capacity to detect and quantify leakage based on optical input reinforces its application for real-time fit assessment, especially in environments where traditional testing resources are unavailable or impractical.

FIGS. 6A and 6B illustrate the performance of system 1000 in detecting leakage across a range of simulated skin pigmentation levels, thereby having different melanin levels. In FIG. 6A, system 1000 was applied to a lighter skin pigmentation, while FIG. 6B shows system 1000 applied to a darker skin pigmentation. Despite the substantial difference in simulated melanin content, system 1000 produced highly consistent leakage visualizations in both cases, with similar spatial patterns, pixel intensities, and computed leakage areas. This result reflects a fundamental advantage of the infrared spectral range selected for use in system 10. Melanin, the primary pigment responsible for variation in human skin tone, exhibits strong absorption in the ultraviolet and visible light spectra, with peak absorption near 335 nm. However, absorption by melanin drops off steeply above 700 nm and becomes negligible in the near-infrared range. System 10 operates with infrared light in the range of approximately 850 nm to 940 nm, a window specifically chosen for its low interaction with melanin. As such, reflectance measurements acquired by camera 106 are unaffected by melanin density, allowing for consistent detection sensitivity across users with varying skin tones. By leveraging this spectral property, system 1000 ensures that the accuracy and reliability of its leakage detection output, such as subtracted image 26 and threshold image 28, are not biased by user phenotype. Thus system 1000 is compatible with various skin tones making it well suited for deployment in demographically diverse populations and eliminates the need for user-specific calibration based on complexion.

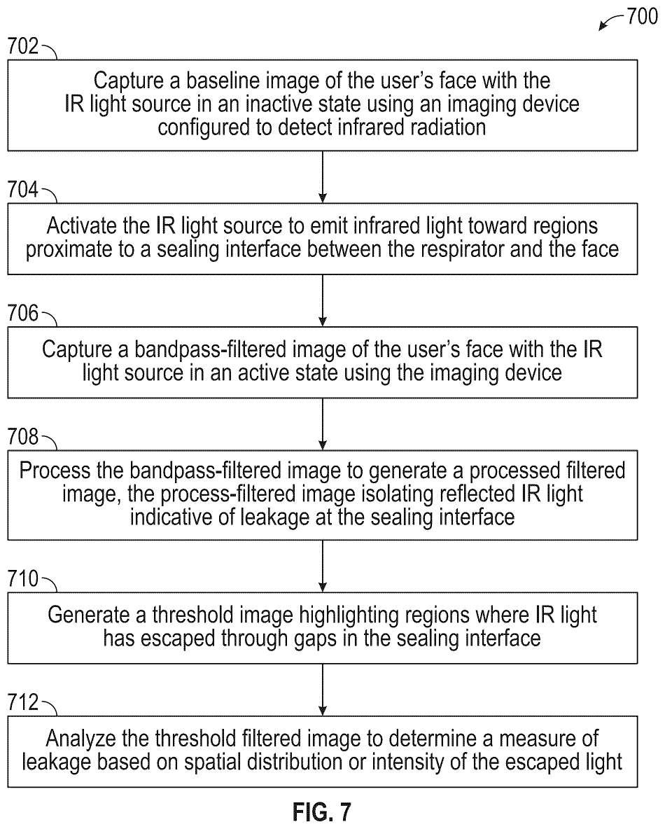

FIG. 7 illustrates a method 700 for detecting leakage from a disposable filtering facepiece respirator using infrared (IR) light reflectance. The method is implemented using system 1000, which includes an IR LED-equipped respirator system 10, an imaging system 400, and a controller 200 with onboard processing and analysis capabilities. Method 700 allows for rapid, non-invasive, and demographically robust leakage detection without reliance on aerosol-based fit testing or user-reported feedback.

At step 702, a first, baseline image 20 of the user's face 102 is captured with the IR light source in an inactive state. The imaging system 400, including camera 106, is configured to detect IR radiation and may optionally include a bandpass optical filter 108 that transmits only wavelengths within the emission spectrum of the IR source. The baseline image 20 represents the reflectance and lighting profile of the face 102 and respirator 100 under ambient conditions, capturing any background illumination, internal mask reflections, and surface geometry without the influence of active IR illumination. This image establishes a reference condition for subsequent image processing (e.g., subtraction) and is critical for isolating the IR leakage signal in downstream analysis.

At step 704, the IR light source 104 is activated to emit infrared light toward regions proximate to the sealing interface 101 of the respirator 100 and the user's face 102. The IR light source 104 may comprise one or more IR LEDs configured to emit light in a wavelength range of approximately 850 to 940 nanometers. This spectral range is selected to maximize signal transmission through air gaps while minimizing absorption by human skin and melanin, thereby enabling consistent reflectance profiles across users with different skin tones. The emitted IR light radiates outward from within the mask cavity, illuminating both well-sealed and leaky regions of the sealing surface.

At step 706, two separate images may be captured with the IR LED in the active state, an unfiltered image 22 and a bandpass-filtered image 24. The unfiltered image 22 captures the scene with IR illumination, including both ambient and reflected IR light, without spectral isolation. The bandpass-filtered image 24, by contrast, is captured using the IR-sensitive camera 106 in conjunction with the bandpass optical filter 108 and isolates only the light in the IR emission range of the LED 104. The unfiltered image 22 highlights both background and reflected IR leakage, while the bandpass-filtered image 24 provides enhanced contrast for leakage visualization by removing most ambient and visible light. In each case, the zone of illumination 110 corresponds to localized reflectance due to leakage at the sealing interface. In some aspects, the unfiltered image 22 and baseline image 20 are processed using pixel-wise processing to generate a differential image, which removes non-informative ambient signal and highlights only the IR reflectance associated with leakage. This differential image may optionally be used to support interpretation, quantification, or visualization of leakage zones.

At step 708, the bandpass-filtered image 24 is further processed to generate a processed filtered image 26. In an aspect, digital masking or segmentation is applied to bandpass-filtered image 24 to exclude areas where IR light transmits through the respirator material itself, yielding a cleaner representation of IR light that escapes only through leaks in the sealing surface. The processed filtered image 26, in turn, isolates the zone of illumination 110 with high spatial specificity and forms the basis for subsequent thresholding and leakage quantification. The controller 200 may apply algorithms configured to enhance image contrast, suppress background signal, and prepare the processed filtered image 26 for further diagnostic analysis.

At step 710, the controller 200 generates a threshold image that highlights regions of significant IR leakage based on the processed filtered image 26. In one embodiment, an intensity threshold is applied to processed filtered image 26 to produce a threshold image 28. The thresholding operation identifies pixels exceeding a predefined reflectance intensity, corresponding to areas where IR light escaped through gaps in the sealing surface 101. The threshold image 28 may be rendered in binary, grayscale, or pseudocolor, and provides a visual map of leakage zones 110. The resulting processed image facilitates interpretation by end users, clinicians, or fit test operators and may be displayed via a user interface or transmitted to a remote monitoring platform.

At step 712, the processed image is further analyzed to determine a quantitative measure of leakage. This may include calculating the total pixel count above the intensity threshold within a region of interest or determining the proportion of such pixels relative to the total facial area captured in the image. In some embodiments, system 1000 uses camera calibration data to convert pixel dimensions into physical area, enabling reporting of leakage in terms of square millimeters or percentage of the sealing surface 101. The resulting output may be expressed as a leak percentage, fit quality score, or diagnostic index and may be used in real time to assess respirator performance. Because the method uses a wavelength band of 850-940 nm, which exhibits minimal absorption by melanin, the measurement remains robust and consistent across individuals with different skin pigmentation.

Method 700 may be executed on a local embedded controller 200, such as a portable computing device or edge processor, or optionally via cloud-based platforms. The method is compatible with a range of mask types, user demographics, and environmental conditions, and may be incorporated into training, deployment, or compliance workflows for occupational or clinical respiratory protection.

In some aspects, the system 1000 and method 700 are configured to detect leakage from multiple users simultaneously. The imaging system 400 may be positioned to capture a shared field of view encompassing a plurality of respirator wearers, and the controller 200 may analyze image data corresponding to each user in parallel. This capability enables concurrent evaluation of respirator fit across a group of individuals, such as in clinical, occupational, or public settings, using a single camera system, thereby supporting large-scale screening and compliance monitoring.

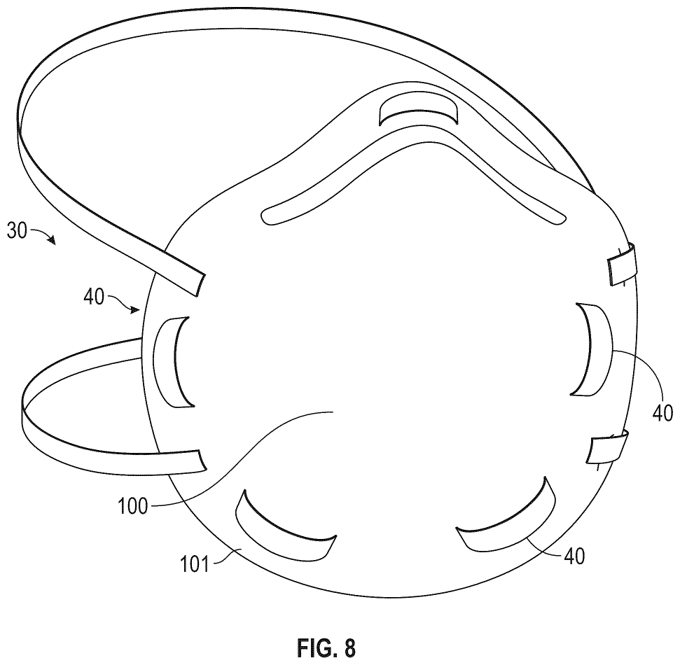

Referring to FIG. 8, an aspect of the present disclosure illustrates a colorimetric leak detection system 30 integrated into a disposable filtering facepiece respirator 100. The colorimetric leak detection system 30 includes a plurality of chemical indicators 40 disposed around the outer periphery of the respirator 100, external to and adjacent to the sealing surface 101. The sealing surface 101 defines the intended contact region between the respirator 100 and the user's face, including the nasal bridge, cheeks, and chin. In this embodiment, the chemical indicators 40 are positioned on the outer surface of the respirator 100, outside the user's direct line of sight, such that they remain visible to others, such as coworkers or supervisors, while avoiding skin contact with the wearer. This allows for real-time detection of leakage events during use, particularly in scenarios involving facial movement, improper donning, or prolonged wear. In environments such as surgical suites, laboratories, or field operations, the external visibility of the indicators allows bystanders to quickly recognize seal failure and take corrective action, thereby enhancing overall safety and reducing the risk of unrecognized respiratory exposure.

Each chemical indicator 40 comprises a filament, fiber bundle, or porous material impregnated with a pH-sensitive dye. In one aspect, phenol red is used as the colorimetric agent, although other acid-base indicators may be substituted depending on the desired sensitivity profile. Phenol red exhibits a distinct and continuous color transition across a pH range of approximately 6.8 to 8.2, shifting from yellow-orange under acidic conditions to deep red or magenta under more alkaline conditions. The chemical formulation is designed to respond to exposure to carbon dioxide (CO₂), which is present in high concentration in exhaled breath but is nearly absent in ambient atmospheric air. When turbulent expiratory airflow escapes through a breach in the seal of the respirator 100, often characterized by high Reynolds number and directional instability, carbon dioxide-rich breath passes over the adjacent indicator 40, where it dissolves into the moisture naturally carried in exhaled air. The dissolved CO₂ forms carbonic acid, releasing hydrogen ions that reduce the local pH of the indicator environment. This local pH change causes the dye in each indicator 40 to undergo a visible color shift, thereby providing an optical signal of leakage.

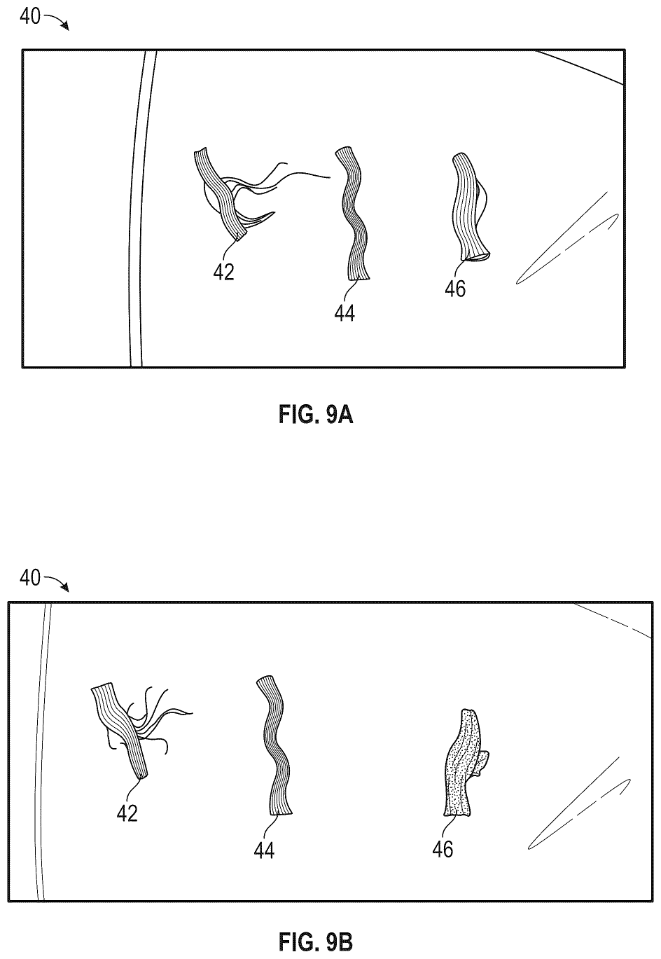

FIGS. 9A and 9B depict the performance of colorimetric leak detection system 30 in a laboratory setting. FIG. 9A shows three untreated indicator fibers 42, 44, and 46, all with similar baseline coloration prior to exposure. FIG. 9B shows the same three indicators after controlled exposure to humidified carbon dioxide gas. Fiber 42 remains unchanged, fiber 44 shows minimal response, and fiber 46 undergoes a distinct color change from yellow to red, indicating a higher level and/or longer duration of CO₂ exposure. The degree of color change is a function of the concentration of hydrogen ions and therefore reflects the persistence and intensity of the respirator leak at the corresponding location on the respirator.

In some aspects of the colorimetric leak detection system 30, the chemical indicators 40 are tuned to resist false positives by incorporating buffering agents or adjusting the pH response curve. Thus, a threshold for activation can be defined, such that brief or transient leaks, such as those occurring during a single breath, may not trigger a visible response, while persistent or repeated leakage will result in a clearly observable color change. The ability to tune this sensitivity allows system 30 to be deployed in clinical, occupational, or field environments with variable operational thresholds for when a mask should be replaced. To maintain wearer safety and comply with biocompatibility requirements, the plurality of chemical indicators 40 are separated from the skin-contacting surface by a waterproof barrier. This barrier prevents direct exposure of the user to the chemical formulation, even under conditions of humidity or prolonged wear. The plurality of chemical indicators 40 are therefore situated just beyond the sealing surface 101, such that only expiratory gas escaping through peripheral leakage, not gas filtered through the mask or absorbed through the skin, contacts the sensing material.

The colorimetric leak detection system 30 operates without electrical power, sensors, or onboard electronics, enabling its use in low-resource environments and reducing cost and complexity. The chemical indicators 40 function solely through passive exposure to ambient conditions and expired breath, allowing for continuous monitoring of seal integrity over the duration of respirator wear. This passive functionality also allows system 30 to be integrated into existing disposable respirator designs without modification to power supply, airflow resistance, or bulk.

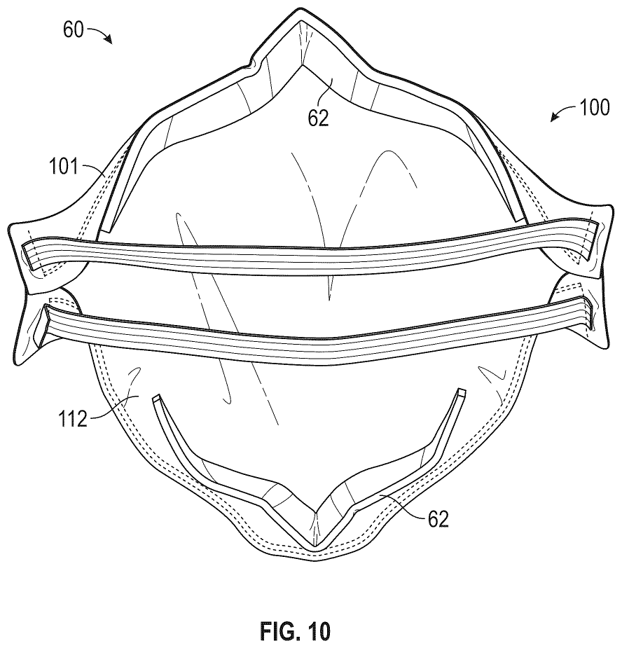

Referring to FIG. 10, an aspect of the present disclosure illustrates a sealing enhancement system 60 integrated into a disposable filtering facepiece respirator 100. The sealing enhancement system 60 includes at least one adhesive segment 62 positioned on the interior surface 112 of the respirator 100 adjacent to the sealing surface 101. The adhesive segment 62 comprises a double-sided medical-grade adhesive tape having a peel-away liner configured to preserve adhesion until the moment of application. Prior to donning the respirator 100, the user may remove the peel-away liner from each adhesive segment 62 and press the respirator 100 into position, allowing the exposed adhesive surface of the adhesive segment 62 to form a continuous seal against the skin.

At least one adhesive segment 62 is disposed on the inside surface 112 of respirator 100 at points where leakage is most likely to occur during normal use, including the nasal bridge, nasolabial folds, and chin. These regions often present challenges in achieving an airtight seal where standard malleable nose clips or elastic straps. In particular, the peel-away configuration allows for a simple and hygienic method of activating the adhesive segment 62 immediately before use. Once in contact with skin, the adhesive segment 62 forms a secure but removable bond that enhances sealing performance without irritating the wearer or altering the structure of the respirator 100. The adhesive segment 62 accommodates a wide range of nasal morphologies by conforming to local geometry upon application, filling minor surface irregularities and creating a more complete barrier to air leakage.

The configuration of the sealing enhancement system 60 is configured to be compatible with users that have non-conventional facial anatomies. For example, individuals exhibiting increased nasal protrusion, broader nasal breadth, lower nasal root height, or other non-normative facial geometries may struggle to achieve proper fit with standard disposable respirators. In such cases, the adhesive segment 62 provides a supplemental sealing mechanism that adapts to individual variation without requiring custom fabrication. By forming a bond between the sealing surface 101 and the user's facial anatomy, the sealing enhancement system 60 mitigates air gaps that would otherwise permit unfiltered airflow. In an aspect, the adhesive segment 62 is biocompatible and formulated for use on intact human skin for extended durations. Biocompatible adhesives such as medical-grade acrylic adhesives are used to minimize the risk of dermatologic irritation, sensitization, or allergic reaction. Accordingly, the sealing enhancement system 60 is well suited for deployment in clinical, industrial, and emergency settings, and may also improve respirator fit for adolescents or individuals underrepresented in conventional anthropometric datasets.

In some aspects, the sealing enhancement system 60 may be used in conjunction with the leakage detection capabilities of system 1000 and system 10. For example, adhesive segments 62 may be applied prior to image capture to improve fit and reduce leakage, thereby facilitating more accurate diagnostics and minimizing false positives during reflectance-based leakage analysis. In some aspects of the present disclosure, the sealing enhancement system 60 and system 1000 for detecting leakage in a disposable may be integrated in use to further support real-time fit validation or iterative adjustment of adhesive placement during respirator donning.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different example embodiments provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A method for detecting leakage from a disposable respirator, comprising:
placing a respirator comprising an infrared (IR) light source on a user's face;
capturing a baseline image of the user's face with the IR light source in an inactive state using an imaging device configured to detect infrared radiation;
activating the IR light source to emit infrared light toward regions proximate to a sealing interface between the respirator and the face;
capturing a bandpass-filtered image of the user's face with the IR light source in an active state using the imaging device;
processing the bandpass-filtered image to generate a processed filtered image, the processed filtered image isolating reflected IR light indicative of leakage at the sealing interface;
generating a threshold image highlighting regions where IR light has escaped through gaps in the sealing interface; and
analyzing the threshold filtered image to determine a measure of leakage based on spatial distribution or intensity of the escaped light.

2. The method of claim 1, wherein the infrared light source comprises one or more infrared light-emitting diodes (IR LEDs) configured to emit light in a wavelength range of about 850 to 940 nanometers.

3. The method of claim 2, wherein the wavelength range of approximately 850 to 940 nanometers is selected to be insensitive to melanin content, thereby enabling consistent detection of infrared reflectance across users with varying skin pigmentation.

4. The method of claim 1, wherein the imaging device includes a bandpass optical filter configured to selectively transmit infrared light in the same wavelength range as the IR light source.

5. The method of claim 1, further comprising capturing an unfiltered image of the user's face with the IR light source in an active state using the imaging device.

6. The method of claim 5, further comprising processing the baseline image and the unfiltered image by performing pixel-wise processing to generate a differential image representing localized reflectance changes.

7. The method of claim 1, further comprising applying an intensity threshold to the process-filtered image to generate the threshold image highlighting regions exceeding a predefined leakage threshold.

8. The method of claim 1, further comprising calculating a leakage percentage by dividing a number of pixels above a defined intensity threshold within the highlighted regions by a total number of pixels within the highlighted regions.

9. The method of claim 1, further comprising displaying the processed filtered image on a user interface during use to visually indicate leakage location and severity.

10. The method of claim 1, wherein the IR light source is configured within the inside surface of the respirator.

11. The method of claim 1, wherein the measure of leakage comprises a computed fit score, leak index, or qualitative diagnostic label based on predefined criteria.

12. The method of claim 1, further comprising
detecting infrared light leakage from a plurality of respirators worn by multiple users within a shared field of view; and
concurrently evaluating a measure of leakage for each of the plurality of respirators.

13. A system for detecting leakage from a disposable respirator, comprising:
an infrared (IR) light source located within a respirator and configured to emit IR light toward a sealing interface between the respirator and a user's face;
an imaging device configured to capture a baseline image of the user's face with the IR light source in an inactive state and a bandpass-filtered image with the IR light source in an active state;
a processor; and
a memory, including instructions stored thereon, which when executed by the processor cause the system to:
process the bandpass-filtered image to identify a zone of illumination, wherein the zone of illumination is isolated IR light indicative of leakage through the sealing interface;
generate a processed filtered image highlighting regions of IR light escape; and
analyze the processed filtered image to determine a measure of leakage based on the spatial distribution or intensity of the escaped IR light.

14. The system of claim 12, wherein the infrared (IR) light source comprises one or more infrared light-emitting diodes (IR LEDs) configured to emit light in a wavelength range of about 850 to 940 nanometers.

15. The system of claim 14, wherein the wavelength range of approximately 850 to 940 nanometers is selected to minimize absorption by melanin, thereby enabling consistent detection of infrared reflectance across users with varying skin pigmentation.

16. The system of claim 13, wherein the imaging device includes a bandpass optical filter configured to selectively transmit infrared light in the same wavelength range as the IR light source.

17. The system of claim 13, wherein the processor is further configured to perform pixel-wise processing between the baseline image and an unfiltered image to generate a differential image representing localized IR reflectance changes, wherein the unfiltered image is of the user's face with the IR light source in an active state.

18. The system of claim 13, wherein the processor is further configured to apply an intensity threshold to the processed filtered image to generate a threshold image highlighting regions exceeding a predefined leakage threshold.

19. The system of claim 13, wherein the processor is further configured to calculate a leakage percentage by dividing a number of pixels above a defined intensity threshold within the zone of illumination by a total number of pixels within the zone of illumination.

20. The system of claim 13, wherein the IR light source is located along an inner surface of the respirator adjacent to the sealing interface.

21. The system of claim 13, wherein the measure of leakage comprises a computed fit score, leak index, or qualitative diagnostic label based on predefined criteria.

22. The system of claim 13, wherein the processor is further configured to analyze the processed image independently of user skin pigmentation by leveraging spectral insensitivity of the selected IR wavelength band to melanin content.
